(19) United States
(12) Patent Application Publication
(10) Pub. No.: US 2004/0133017 A1
(43) Pub. Date: Jul. 8, 2004

(54) Process for the preparation of citalopram

(76) Inventors: Reddy Muddasani Pulla, Hyderabad (IN); Chowdary Venkaiah, Hyderabad (IN)

Correspondence Address: MERCHANT & GOULD PC, P.O. BOX 2903, MINNEAPOLIS, MN, 55402-0903, US

(21) Appl. No.: 10/468,973

(22) PCT Filed: Feb. 11, 2002
(86) PCT No.: PCT/IN02/00023

(30) Foreign Application Priority Data
Feb. 22, 2001 (IN) 157/MAS/2001

(51) Int. Cl. C07D 307/87

(52) U.S. Cl. 549/467

(57) ABSTRACT

This invention discloses an improved process for the preparation of citalopram of the formula III which comprises (i) preparing the compound of the formula VIII by reducing an unisolable magnesium salt of a benzophenone derivative of the formula V using sodium borohydride in the presence of a protic solvent, (ii) reacting the compound of the formula VIII obtained in step (i) with an acid catalyst in a non-polar solvent to obtain a compound of the formula I, (iii) reacting the compound of the formula I obtained in step (ii) with copper (I) cyanide in a polar solvent medium and isolating the resulting cyano compound, by recrystallization by using polar and/or alcoholic solvents to obtain the compound of the formula II and (III) reacting the resulting compound of the formula II by conventional methods to form citalopram of the formula III. Citalopram is widely used as an antidepressant.

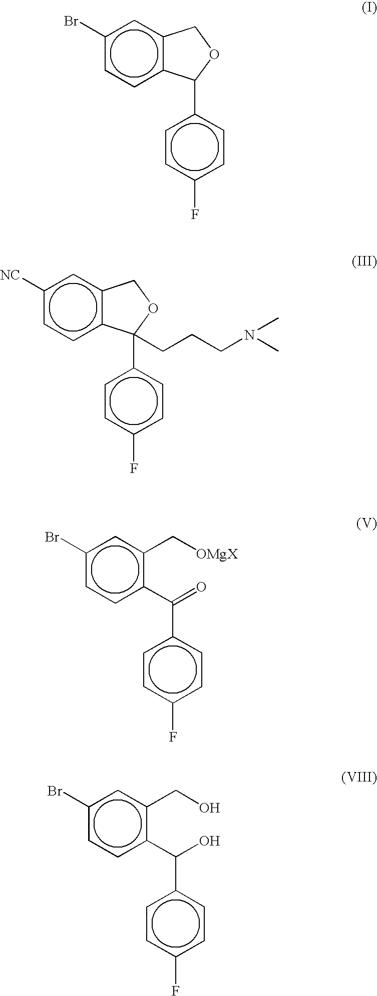

The present invention relates to an improved process for the preparation of citalopram. It is well known that citalopram is a good antidepressant which is widely used. The present invention also relates to an improved process for the

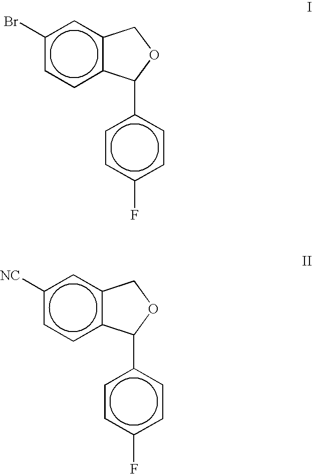

preparation of the intermediates of the formula I & II which are useful for the preparation of citalopram, a well known antidepressant.

The compounds of the Formulae-I & II are key intermediates used in the synthesis of known antidepressant drug 1-(3-dimethylaminopropyl)-1-(4¹-fluorophenyl)-1,3-dihydroisbenzofuran-5-carbonitrile (citalopram), of the Formula-III) and its pharmaceutically acceptable acid addition salts thereof.

The process for the preparation of antidepressant citalopram and its pharmaceutoical properties were first disclosed in DE Patent no. 2,657,013 (1977) corresponding to U.S. Pat. No. 4,136,193 (1979). Subsequently it was also disclosed in GB patent no.1,526,331 (1978).

The basic process for the preparation of citalopram described in the above referred patents involves two major routes illustrated in Scheme-1 and Scheme-2. Major difference in these two routes is introduction of dimethylaminopropyl side chain at an early stage (Scheme-1) or at a later stage (Scheme-2).

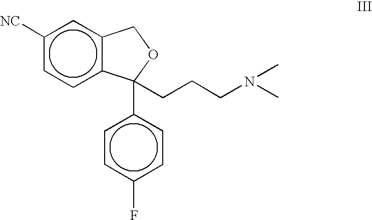

In the first route, 5-bromophthalide of the Formula-IV is reacted with p-fluorophenylmagnesium bromide to get a benzophenone derivative of the formula Formula-V. This benzophenone derivative is reacted with 3-N,N-dimethylaminopropylmagnesium chloride to get the dihydroxy intermediate of the Formula-VI. Cyclization with an acid catalyst resulted in the formation of phthalane derivative of the Formula-VII. This bromophthalane derivative is reacted with copper cyanide to get the citalopran base of the Formula III.

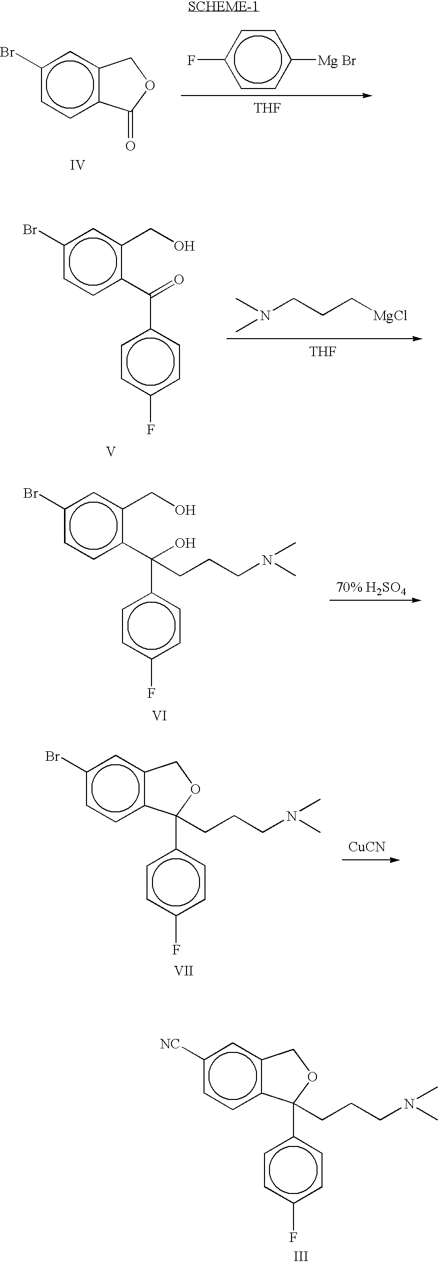

In the second route, 5-bromophthalide of the formula-IV is reacted with p-fluorophenyl-magnesium bromide to get the corresponding benzophenone derivative of the Formula-V. This compound is reduced with lithium aluminium hydride to get the dihydroxy compound of the Formula-VIII, which is cyclized with an acid catalyst to get the phthalane derivative of the Formula-I. The bromo group is replaced with a cyano group and alkylated with the required side chain to get the citalopram base.

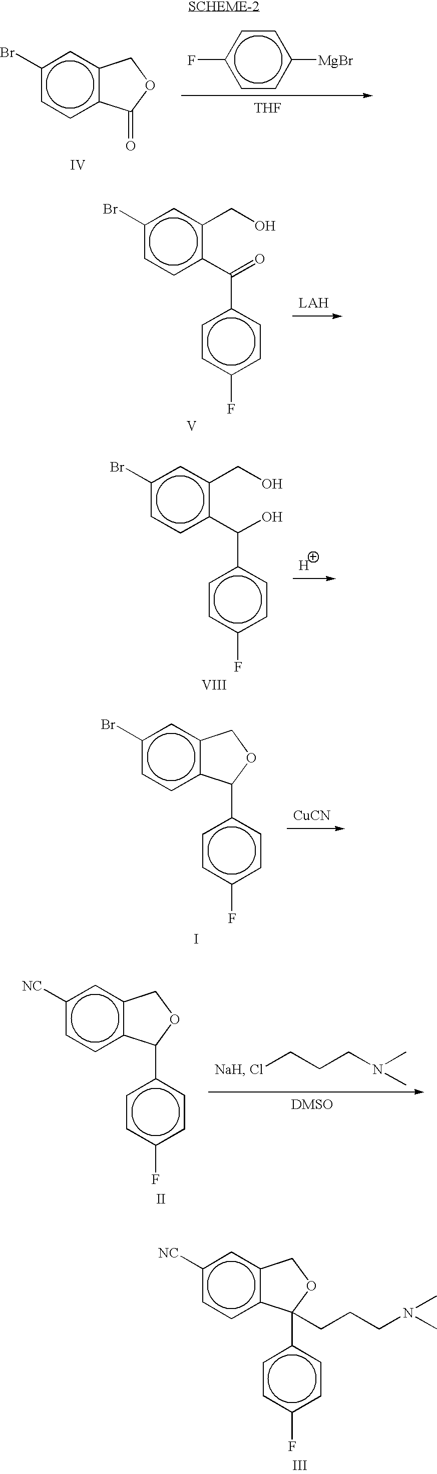

Bogeso (EP patent no.171,943, corresponding to U.S. Pat. No. 4,650,884) has indicated that the methods described in the above patents for the preparation of citalopram possess some problems in the scale-up to commercial production.

In an attempt to develop a shorter route for the preparation of citalopram and to avoid the risk involved in the metalation step used previously, Bogeso started with 5-cyanophthalide of the Formula-IX and surprisingly found that cyano group survived the cyclization step where 70% sulfuric acid was used at 80° C. temperature (Scheme-3).

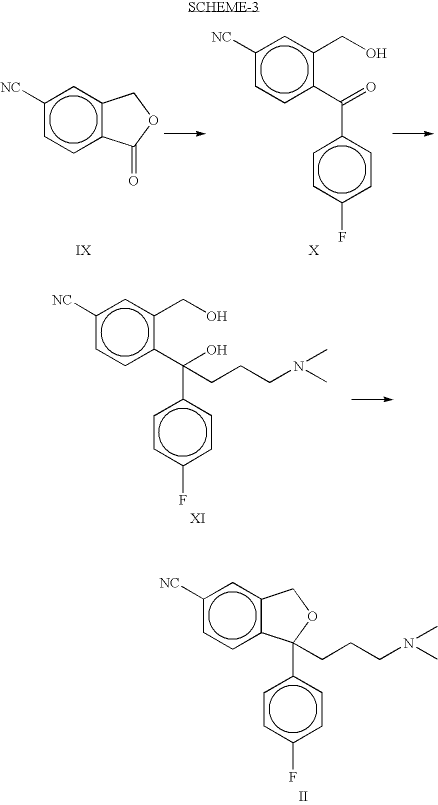

Further processes have been disclosed in international patent application nos. WO 981019511, WO 98/019512, WO 98/019513, WO 99/030548, WO 00/011926, WO 00/013648, and WO 00/023431. International patent application no. WO 98/019511 discloses a process for the manufacture of citalopram wherein a compound of the Formula-X was reduced with sodium borohydride to get a compound of the Formula-XII. However, yield is only 40% and large quantity (˜50 times) of alcohol was used. This compound of the Formula-XII is subjected to ring closure and the resulting 5-substituted dihydroisobenzofuran derivative is converted to the corresponding 5-cyano derivative and alkylated with (3-dimethylamino) propyl halogenide to obtain citalopram.

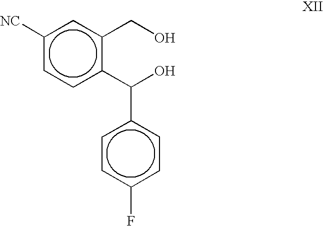

WO 98/019512 and WO 98/019513 relate to methods wherein a 5-amino-, 5-carboxy- or 5-(sec-aminocarbonyl) phthalide is subjected to two successive Grignard reactions, ring closure and conversion of the resulting 1,3-dihydroisobenzofuran derivative to the corresponding 5-cyano compound, i.e. citalopram.

International patent application no. 99/030548 discloses a process for the preparation of citalopram wherein cyano group was introduced from the corresponding 5-aldehyde analogue of citalopram.

International patent application nos. WO 00/011926 and WO 00/013648 disclose an improved process for the preparation of citalopram wherein 5-halogen (Cl or Br) analogue of citalopram is activated by using palladium or nickel complex catalyst to introduce the corresponding cyano group present in citalopram.

International patent application no. WO 00/023431 discloses a process for introduction of cyano group present in citalopram via the corresponding 5-oxazolyl analogue of citalopram.

A major drawback in the scale up to commercial production of citalopram by following the original patent process (disclosed in U.S. Pat. No. 4,136,193) is removal of impurities present in citalopram to an acceptable level of pharmaceutical quality. Methods followed to improve the quality of citalopram are either by chemical purification (via acid addition salt where ever applicable) or by high vacuum distillation. Chemical method does not seem to remove the impurities up to the acceptable level because some of the impurities like compound of Formula-VI, Formula-VII or Formula-XIII have similar salt formation properties with an acid.

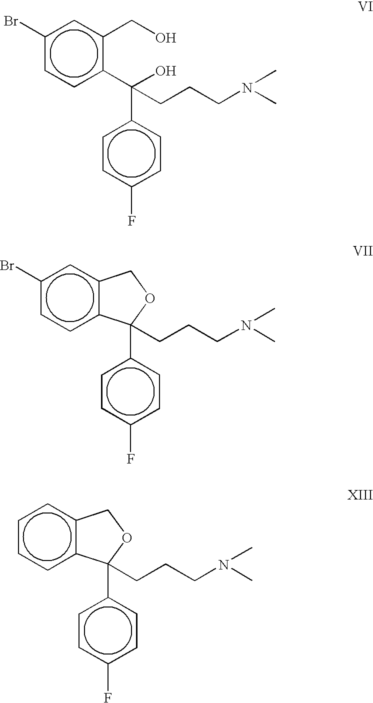

All the intermediates involved in the original patent for the preparation of citalopram have very high boiling point (˜200° C. at <0.1 mm Hg) and are sensitive to overheating. This is also a major drawback in commercialising the process.

Second route of the original patent for the preparation of citalopram involves purification of intermediate compounds of the Formula I and II by high vacuum distillation (180-200° C. at <0.1 mm Hg). This process is also practically difficult for a commercial production. Also, this route involves handling of a costly and hazardous reagent, lithium aluminium hydride.

Third and simplified route (disclosed in EP Patent no. 171,943) for the preparation of citalopram involves the introduction of 5-cyano group present in citalopram at the beginning itself. This route also has major drawback of removal of impurities present in citalopram. Repeated recrystallization technique was described in making pharmaceutically acceptable quality citalopram. Also, there is a considerable loss if required product (citalopram) in this technique.

All other international patents published between 1998 and 2000 are involving with various methods to introduce 5-cyano group from different functional groups. All these methods are focusing on new chemistry and are not adaptable for commercial production.

Citalopram has become a well known antidepressant drug that has now been on the market and has shown great promise as a valuable antidepressant drug with few side effects. Keeping in view of the difficulties in commercialization of the above mentioned processes for the preparation of citalopram, we aimed to develop a simple and economical process for commercial production of citalopram.

We observed that a promising approach for such a process is to (a) improve the quality of one or more of the isolable intermediates by simple techniques (b) avoid costly and risky reagents like lithium aluminium hydride and (c) minimize the effluents like large quantity of phosphoric acid.

Accordingly the main objective of the present invention is to provide an improved process for the preparation of citalopram of the formula III avoiding the formation of impurities.

Another objective of the present invention is to provide an improved process for the preparation of citalopram with high yield (>90%) and high purity (>99%).

Still another objective of the present invention is to provide an improved process for the preparation of citalopram of the formula III which is simple, economical and environmentally safe.

Another objective of the present invention is to provide an improved process for the preparation of the intermediates of the formulae I & II which are useful for the preparation of citalopram of the formula III.

Yet another objective of the present invention is to provide an improved process for the preparation of intermediates of the formulae I & II which are useful for the preparation of citalopram avoiding the introduction of (3-dimethylamino)propyl side chain present in citalopram at an early stage.

Still another objective of the present invention is to provide an improved process for the preparation of the intermediates of the formulae I & II which are useful for the preparation of citalopram employing a simple crystallization technique.

Further objective of the present invention is to provide an improved process for the preparation of the formulae I & II which are useful for the preparation of citalopram by replacing the costly and hazardous lithium aluminium hydride with simple sodium borohydride and with no involvement of additional steps.

Another objective of the present invention is to provide an improved process for the preparation of the formulae I & II which are useful for the preparation of citalopram by replacing or reducing the acid catalyst used in the cyclization step of the synthesis.

Still another objective of the present invention is to provide an improved process for the preparation of the formulae I & II which are useful for the preparation of citalopram by simplify the process by not involving additional step and avoiding large quantities of solvent (alcohol).

The present invention has been developed based on our finding that if the (3-dimethylamino)propyl side chain present in citalopram is introduced at an early stage, it is difficult to remove the related impurities by conventional methods. Further if a simple crystallization technique for the formation of one or more of the isolable intermediates, it becomes easy to get citalopram with acceptable pharmaceutical quality.

Accordingly the present invention provides an improved process for the preparation of citalopram of the formula III

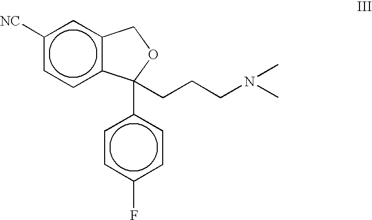

which comprises

(i) preparing the compound of the formula VIII by reducing an unisolable magnesium salt of a benzophenone derivative of the formula V.

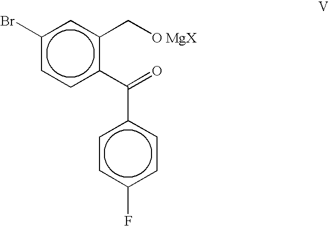

using sodium borohydride in the presence of a protic solvent

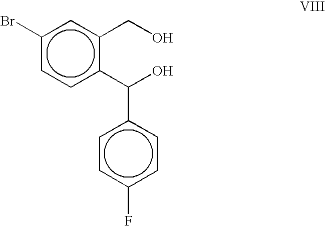

(ii) reacting the compound of the formula VIII obtained in step(i) with an acid catalyst in a non-polar solvent to obtain the compound of the formula I

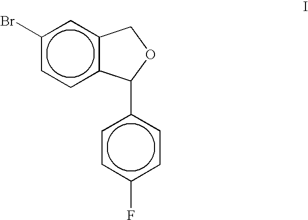

(iii) reacting the compound of the formula I obtained in step (ii) with copper (I) cyanide in a polar solvent and isolating the resulting cyano compound, by recrystallization by using polar and or alcoholic solvents to obtain the compound of the formula II and

(iv) reacting the resulting compound of the formula II by conventional methods to form citalopram of the formula III.

The conversion of the compound of the formula II into compound of the formula III may be effected by reacting the compound of the formula II with a strong base such as NaH, ^{t}BuOK, in a polar solvent such as DMSO, followed by quenching the anion with N,N-dimethylaminopropyl chloride to get citalopram of formula III.

According to a feature of the present invention there is provided an improved process for the preparation of the compound of the formula VIII.

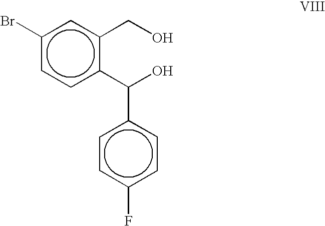

which is useful for the preparation of citalopram of the formula III

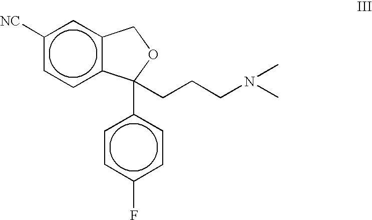

which comprises

(i) reducing an unisolable magnesium salt of a benzophenone derivative of the formula V.

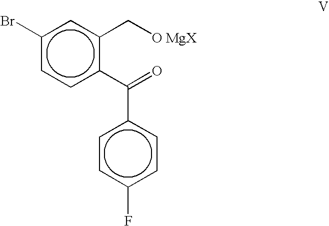

using sodium borohydride in the presence of a protic solvent

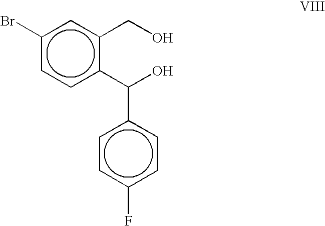

The above process of preparing the compound of the formula VIII has been made the subject matter of our co-pending application Ser. No. ______ which is divided out of this application.

According to another embodiment of the present invention there is provided an improved process for the preparation of intermediate of formula II which is useful for the preparation of citalopram which comprises

(i) reducing an unisolable magnesium salt of a benzophenone derivative of the formula V

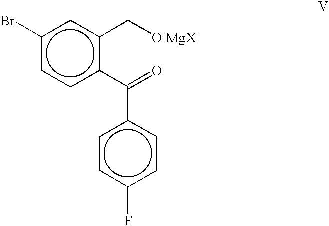

using sodium borohydride in the presence of a protic solvent to obtain a compound of the formula VIII

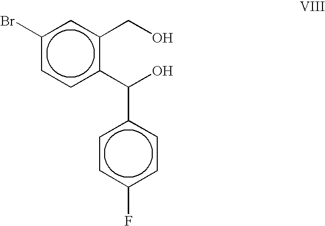

(ii) reacting the compound of the formula VIII obtained in step(i) with an acid catalyst in a non-polar solvent to obtain a compound of the formula I

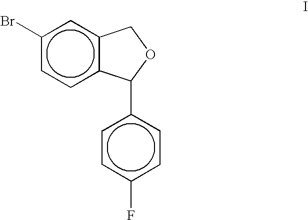

(ii) reacting the compound of the formula I obtained in step (ii) with copper (I) cyanide in a polar solvent medium and isolating the resulting cyano compound, by recrystallization by using polar and or alcoholic solvents to obtain the compound of the formula II.

This process has been made the subject matter for our another co-pending application Ser. No. ______ which is divided out of this application.

According to yet another embodiment of the present invention there is provided an improved process for the preparation of an intermediate of the formula I

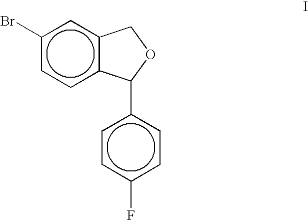

useful for the preparation of citalopram of the formula III which comprises

(i) preparing the compound of the formula VIII by reducing an unisolable magnesium salt of a benzophenone derivative of the formula V

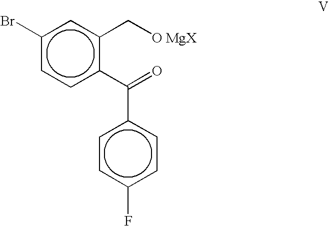

using sodium borohydride in the presence of a protic solvent and

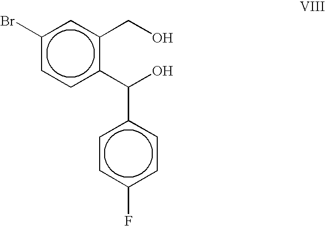

(ii) reacting the compound of the formula VIII obtained in step(i) with an acid catalyst in a non-polar solvent to obtain a compound of the formula I

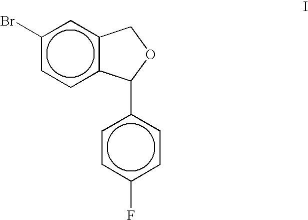

The process of preparing the compound of the formula I has been made the subject matter of yet another co-pending application for U.S. Pat. No. ______ which is also divided out of this application.

The reduction in step (i) may be effected at a temperature in the range of −20° C. to 25° C. preferably at a temperature in the range of 0° C. to 10° C. The protic solvent used in step (i) may be selected from MeOH, ETOH, IPA, t-BuOH and the like.

In an another preferred embodiment of the invention the non-polar solvent such as benzene, toluene, xylene and the like may be used in the step (ii). The acid catalyst such as p-TsOH, H₂SO₄, benzenesulphonic acid and the like may be used.

The crystallization method employed for the isolation of the compound of formula-II consists of dissolving the crude compound of the formula II formed in single solvent like methanol ethanol or isopropanol, or mixed solvent like IPA/MeOK, IPA/DMF, MeOH/DMF, etc. The ratio of the combination may be 4-5:1-3, preferably 3-4:1-2.

The isolated intermediate of formula-II by the process of the present invention is found to be of very high purity (>99% by HPLC) with a melting point of 96-97° C. Further confirmation of the quality was checked by converting this intermediate to the required citalopram hydrobromide salt by known method (U.S. Pat. No. 4,136,193) without requiring any recrystallization process. It is interesting to note that the intermediate of formula-II has got good crystallization property leaving all the impurities in the solvent medium of crystallization.

This simplification has led to the synthesis of this crucial intermediate of the formula II in a very simple and easy to adopt manner suitable for any commercial scale. Also, without any repeated recrystallization techniques, citalopram hydrobromide could be prepared.

The advantage of the invention is that the compound of the formula II can be prepared without isolating the intermediate of the formula I which enhances the yield of the compound of the formula II. Consequently when the process is employed for the preparation of citalopram further increases the yield of citalopram.

The invention is described in detail in the Example given below which are provided only by way of illustration and therefore should not be construed to limit the scope of the invention further illustrated by the following example.

### EXAMPLE 1

### Preparation of Citalopram

### (a) Preparation of 4-bromo-(2-hydroxymethyl)phenyl-(4¹-fluorophenyl)methanol of formula-VIII

The Grignard solution prepared from 90 gr of 4-fluorobromobenzene and 13 gr magnesium turnings in 450 ml of THF was added dropwise to a suspension of 5-bromophthalide (100 gr) in THF (600 ml) at −10 to 0° C. under nitrogen atmosphere. After the addition was over the reaction mixture was stirred at same temperature for another 3 hrs and treated with a slurry of sodium borohydride (25 gr) in 300 ml of IPA keeping the temperature below 10° C. After maintaining for 1 hr at 10° C., reaction was quenched into dil hydrochloric acid (220 ml conc HCl in 1750 ml water). After stirring the reaction mass for 30 min, layers were separated. The aqueous layer was extracted with 3×100 ml of toluene. Combined organic layer was washed with saturated sodium chloride (300 ml) and dried over sodium sulfate. Solvents were removed under vacuum below 60° C. to get the crude compound of the formula VIII (200 gr). This compound is suitable for use in next stage of the process.

### (b) Preparation of 1-(4-fluorophenyl)-5-bromophthalan of formula-I using p-toluene sulfonic acid as catalyst

The crude oily compound of the formula VIII (200 gr) obtained from step (a) above was dissolved in 1000 ml of toluene. To this solution was added 10 gr of p-toluene sulfuric acid and heated to reflux. Water formed in the reaction was removed using Dean-Stark apparatus. When the water formation was over, reaction mass was cooled to room temperature and 1000 ml of water added. After stirring for 30 min organic layer was separated and the aqueous layer extracted with 3×100 ml of toluene. The combined organic layer was washed with 2×250 ml of 5% sodium carbonate solution. Finally the organic layer was washed with saturated sodium chloride. Toluene was removed under vacuum below 60° C. to get the crude compound of the formula I (150 gr) as an oil.

### (c) Preparation of 1-(4-fluorophenyl)-5-cyanophthalan of formula II

To a solution of the compound of the formula I (150 gr) obtained in step (b) above in DMF (360) was added freshly prepared copper (I) cyanide (76 gr). The resulting suspension was slowly heated to reflux temperature and maintained at reflux for 4-5 hrs. After cooling the reaction mass to 40-50° C., aqueous ammonia (200 ml 10% w/v) was added and stirred for 30 min. After filtering off the insoluble salts, layers were separated. The organic layer was washed with 200 ml of dil. ammonia (10% solution). Combined aq. layers were extracted with 100 ml of toluene. to Toluene layers were combined and the solvent distilled off under vacuum at 50-60° C. to give the crude cyano compound of the formula II (120 gr) as a semisolid.

### (d) Purification of 1-4-fluorophenyl)-5-cyanophthalan by recrystallization technique

(i) Recrystallization from IPA.

The crude compound of the formula II(50 gr) obtained in step(c) above was dissolved in 200 ml of IPA by heating to 60-70° C. and treated with 5 gr of charcoal. After filtration, cooling to 20-25° C., it was kept at this temperature for 8-12 hrs. Filtration of the solids and washing with 20-25 ml of IPA gave light yellow crystalline solid (35 gr) m.p. 96-97° C. Purity by HPLC is 98%.

### (e) Preparation of 1-(3-Dimethylaminopropyl)-1-(4-fluorophenyl)-5-cyanophthalan of formula III

A solution of dimsyl sodium in DMSO was prepared by adding 22 gr of 50% sodium hydride in parafin oil to DMSO (1000 ml) at 20-25° C. and slowly heating to 60-65° C. under nitrogen. To this solution at 20-25° C. was added a solution of 1-(4-fluorophenyl)-5-cyanophthalan (100 gr) in DMSO (200 ml) slowly in 2-3 hrs. After maintaining for 15-20 min, a solution of 3-dimethylaminopropylchloride (56 gr) in toluene (120 ml) was slowly added keeping the temperature between 25-30° C. After the addition is over, reaction mixture was maintained at this temperature for 30 min and decomposed by adding 50 ml of methanol. The reaction mixture was poured into 3000 ml of water and extracted with 1000 ml of toluene. Aq. layer was again extracted with 500 ml of toluene. The combined toluene layer was washed with water (500 ml), followed by 2×1000 ml of 20% aqueous acetic acid. The combined aqueous acetic acid layer was neutralized with aqueous ammonia (25%) to get the pH of 7-7.5. After the pH adjustment, 500 ml of isopropyl ether was added and stirred for 15 min. Isopropyl ether layer was separated and the aqueous layer extracted with 2×300 ml of isopropyl ether. The combined isopropyl ether layer was treated with carbon (10 gr) and filtered. The filtrate was distilled off under vacuum below 45° C. to get the compound of the formula III as a light yellow solid (120 gr). mp. 95° C. Purity by HPLC is 99%.

### EXAMPLE 2

### Preparation of Citalopram

### (a) Preparation of 4-bromo-(2-hydroxymethyl)-phenyl-(4¹-fluorophenyl)methanol of formula-VIII

The Grignard solution prepared from 90 gr of 4-fluorobromobenzene and 13 gr magnesium turnings in 450 ml of THF was added dropwise to a suspension of 5-bromophthalide (100 gr) in THF (600 ml) at −10 to 0° C. under nitrogen atmosphere. After the addition was over the reaction mixture was stirred at same temperature for another 3 hrs and treated with a slurry of sodium borohydride (25 gr) in 100 ml of methanol keeping the temperature below 0° C. After maintaining for 1 hr at 10° C., reaction was quenched into dil hydrochloric acid (220 ml conc HCl in 1750 ml water). After stirring the reaction mass for 30 min, layers were separated. The aqueous layer was extracted with 3×100 ml of toluene. Combined organic layer was washed with saturated sodium chloride (300 ml) and dried over sodium sulfate. Solvents were removed under vacuum below 60° C. to get the crude compound of the formula VIII (200 gr). This compound is suitable for use in next stage of the process.

### (b) Preparation of 1-(4-fluorophenyl)-5-bromophthalan of formula-I using benzenesulfonic acid as catalyst

The crude oily compound of the formula VIII (200 gr) obtained from step (a) above was dissolved in 1000 ml of toluene. To this solution was added 10 gr of benzenesulfonic acid and heated to reflux. Water formed in the reaction was removed using Dean-Stark apparatus. When the water formation was over, reaction mass was cooled to room temperature and 1000 ml of water added. After stirring for 30 min organic layer was separated and the aqueous layer extracted with 3×100 ml of toluene. The combined organic layer was washed with 2×250 ml of 5% sodium carbonate solution. Finally the organic layer was washed with saturated sodium chloride. Toluene was removed under vacuum below 60° C. to get the crude compound of the formula I (150 gr) as an oil.

### (c) Preparation of 1-(4fluorophenyl)-5-cyanophthalan of formula II

To a solution of the compound of the formula I (150 gr) obtained in step (b) above in DMAc (300 ml) was added freshly prepared copper (I) cyanide (76 gr). The resulting suspension was slowly heated to 150-160° C. and maintained at that temperature for 4-5 hrs. After cooling the reaction mass to 40-50° C., aqueous ammonia (200 ml, 10% w/v) was added and stirred for 30 min. After filtering off the insoluble salts, layers were separated. The organic layer was washed with 200 ml of dil. ammonia (10% solution). Combined aq. layers were extracted with 100 ml of toluene. Toluene layers were combined and the solvent distilled off under vacuum at 50-60° C. to give the crude cyano compound of the formula II (120 gr) as a semisolid.

### (d) Purification of 1-4-fluorophenyl)-5-cyanophthalan of the formula II by recrystallization technique

(i) Purification by Recrystallization from Methanol.

The crude compound of the formula II (50 gr) obtained in step (c) above was dissolved in 150 ml of refluxing methanol and treated with 5 gr of charcoal. After filtration of carbon, filtrate was cooled to 20-25° C. and maintained for 8-12 hrs. Filtration of the solid and washing the wet cake with 25 ml of methanol gave 25 gr of white crystalline solid. m.p. 97-98° C. purity by HPLC is 99%.

### (e) Preparation of 1-(3-Dimethylaminopropyl)-1-(4-fluorophenyl)-5-cyanophthalan of formula III

To a stirred suspension of 22 gr of sodium hydride (50-55% in parafin oil) in 1000 ml of DMSO at 20-25° C. was added a solution of 1-(4-fluorophenyl)-5-cyanophthalan (100 gr) in DMSO (200 ml) slowly in 2-3 hrs. After maintaining for 15-20 min, a solution of 3-dimethylaminopropylchloride (56 gr) in toluene (120 ml) was slowly added keeping the temperature between 25-30° C. After the addition is over, reaction mixture was maintained at this temperature for 30 min and decomposed by adding 50 ml of methanol. The reaction mixture was poured into 3000 ml of water and extracted with 1000 ml of toluene. Aq. layer was again extracted with 500 ml of toluene. The combined toluene layer was washed with water (500 ml), followed by 2×1000 ml of 20% aqueous acetic acid. The combined aqueous acetic acid layer was neutralized with aqueous ammonia (25%) to get the pH of 7-7.5. After the pH adjustment, 500 ml of isopropyl ether was added and stirred for 15 min. Isopropyl ether layer was separated and the aqueous layer extracted with 2×300 ml of isopropyl ether. The combined isopropyl ether layer was treated with carbon (10 gr) and filtered. The filtrate was distilled off under vacuum below 45° C. to get the compound of the formula III as a light yellow solid (118 gr). m.p. 95° C. Purity by HPLC is 99%.

### EXAMPLE 3

Preparation of Citalopram

### (a) Preparation of 4-bromo-(2-hydroxymethyl)-phenyl-(4¹-fluorophenyl)methanol of formula-VIII

The Grignard solution prepared from 90 gr of 4-fluorobromobenzene and 13 gr magnesium turnings in 450 ml of THF was added dropwise to a suspension of 5-bromophthalide (100 gr) in THF (600 ml) at −10 to 0° C. under nitrogen atmosphere. After the addition was over the reaction mixture was stirred at same temperature for another 3 hrs and treated with a slurry of sodium borohydride (25 gr) in 200 ml of ethanol keeping the temperature below 0° C. After maintaining for 1 hr at 10° C., reaction was quenched into dil hydrochloric acid (220 ml conc HCl in 1750 ml water). After stirring the reaction mass for 30 min, layers were separated. The aqueous layer was extracted with 3×100 ml of toluene. Combined organic layer was washed with saturated sodium chloride (300 ml) and dried over sodium sulfate. Solvents were removed under vacuum below 60° C. to get the crude compound of the formula VIII (200 gr). This compound is suitable for use in next stage of the process.

### (b) Preparation of 1-(4fluorophenyl)-5-bromophthalan of formula I using sulfuric acid as a catalyst

The crude oily compound (200 gr) obtained from Example 3 (a) was dissolved in 1000 ml of toluene and 10 gr of conc. sulfuifc acid was added to this solution. The reaction mixture was heated to reflux and water formed in the reaction was removed azeotropically. After completion of the reaction usual work up gave 150 gr of the compound of the formula II as an oil.

### (c) Preparation of 1-(4-fluorophenyl)-5-cyanophthalan of formula II

To a solution of the compound of the formula I (150 gr) obtained in step (b) above in pyridine (150 ml) was added freshly prepared copper (1) cyanide (76 gr). The resulting suspension was slowly heated to reflux temperature and maintained at reflux for 4-5 hrs. After cooling the reaction mass to 40-50° C., aqueous ammonia (200 ml 10% w/v) was added and stirred for 30 min. After filtering off the insoluble salts, layers were separated. The organic layer was washed with 200 ml of dil. ammonia (10% solution). Combined aq. layers were extracted with 100 ml of toluene. Toluene layers were combined and the solvent distilled off under vacuum at 50-60° C. to give the crude cyano compound of the formula II (120 gr) as a semisolid.

### (d) Purification of 14-fluorophenyl)-5-cyanophthalan of the formula II by recrystallization technique

(i) Recrystallization from IPA-DMF.

The crude compound of the formula II (150 gr) obtained in step (c) above was dissolved in 100 ml of IPA-DMF (80:20) at 50-60° C. and treated with 5 gr of active charcoal. After filtration of the charcoal, filtrate was cooled to 10-15° C. and maintained for 3-4 hrs at this temperature. The solids formed were filtered and the wet cake washed with 20 ml of IPA to get white crystalline solid. m.p. 97-98° C. Purity by HPLC is 98.5%.

### (e) Preparation of 1-3-Dimethylaminopropyl)-1-(4-fluorophenyl)-5-cyanophthalan of formula III

To a stirred solution of 59 gr of potassium t-butoxide in DMSO (1000 ml) at 20-25° C. was added a solution of 1-(4-fluorophenyl)-5-cyanophthalan (100 gr) in DMSO (200 ml) slowly in 2-3 hrs. After maintaining for 15-20 min, a solution of 3-dimethylaminopropylchloride (56 gr) in toluene (120 ml) was slowly added keeping the temperature between 25-30° C. After the addition is over, reaction mixture was maintained at this temperature for 30 min and decomposed by adding 50 ml of methanol. The reaction mixture was poured into 3000 ml of water and extracted with 1000 ml of toluene. Aq. layer was again extracted with 500 ml of toluene. The combined toluene layer was washed with water (500 ml), followed by 2×1000 ml of 20% aqueous acetic acid. The combined aqueous acetic acid layer was neutralized with aqueous ammonia (25%) to get the pH of 7-7.5. After the pH adjustment, 500 ml of isopropyl ether was added and stirred for 15 min. Isopropyl ether layer was separated and the aqueous layer extracted with 2×300 ml of isopropyl ether. The combined isopropyl ether layer was treated with carbon (10 gr) and filtered. The filtrate was distilled off under vacuum below 45° C. to get the compound of the formula III as a light yellow solid (100 gr). m.p. 95° C. Purity by HPLC is 99%.

### Advantages of the Present Invention

1. Replacing lithium aluminium hydride with sodium borohydride is very much. cost effective and free of any hazardous nature.

2. Simple crystallization method for the cyano compound of the Formula-II has avoided the high vacuum distillation of the corresponding bromo derivative of the Formula-I.

3. The resulting compound of the formula III is produced in high yield (88%) and of high purity (99%).

4. The process is adaptable to any commercial scale and environmentally safe and economical.

We claim:

1. An improved process for the preparation of citalopram of the formula III 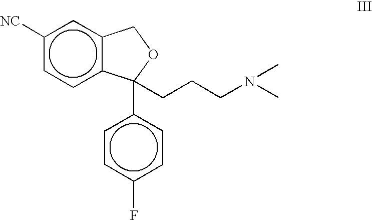
which comprises
(i) preparing the compound of the formula VIII by reducing an unisolable magnesium salt of a benzophenone derivative of the formula V. 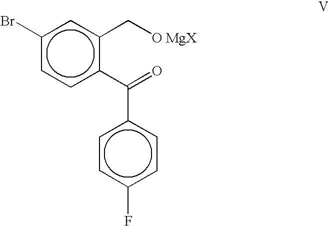
using sodium borohydride in the presence of a protic solvent 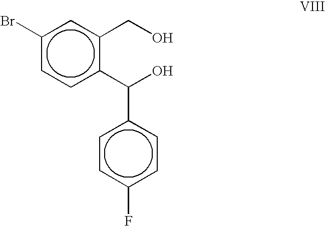
(ii) reacting the compound of the formula VIII obtained in step(i) with an acid catalyst in a non-polar solvent to obtain a compound of the formula I 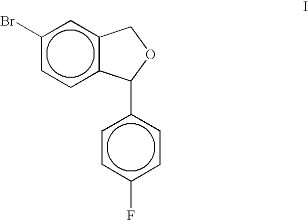
(iii) reacting the compound of the formula I obtained in step (ii) with copper (1) cyanide in a polar solvent medium and isolating the resulting cyano compound, by re-crystallization by using polar and/or alcoholic solvents to obtain the compound of the formula II and
(iv) reacting the resulting compound of the formula II by conventional methods to form citalopram of the formula III

2. A process as claimed in claim 1 wherein protic solvent such as MeOH, EtOH, IPA, t-BuOH, preferably methanol, is used in step (i).

3. A process as claimed in claims 1 & 2 wherein non-polar solvent such as benzene, toluene, xylene, cyclohexane, preferably toluene, is used in step (ii).

4. A process as claimed in claims 1 to 3 wherein the catalyst such as benzenesulfonic acid, p-toluenesulfonic acid, sulfuric acid, preferably p-TsOH, is used in step (iii).

5. A process as claimed in claims 1 to 4 wherein the solvent used for recrystallization in step (ii) is selected from methanol, IPA, ethanol with or without DMF, or a combination thereof.

6. A process as claimed in claim 5 wherein the solvent used for recrystallilation is a combination of IPA with DMF.

7. A process as claimed in claim 6 wherein the ratio of IPA & DMF used ranges from 5-6:1-3 preferably in the range 3-4:1-2.

8. An improved process for the preparation of citalopram of the formula III substantially as herein described with reference to the Example 1 to 3.
